(19) United States
(12) Patent Application Publication
(10) Pub. No.: US 2026/0088133 A1
(43) Pub. Date: Mar. 26, 2026

(54) REPRESENTATION LEARNING MODELS FOR IMPROVED GENOMICS

(71) Applicant: GOOGLE LLC, Mountain View, CA (US)

(72) Inventors: Taedong YUN, Boston, MA (US); Justin Thomas COSENTINO, Philadelphia, PA (US); Babak BEHSAZ, Boston, MA (US); Andrew Walker CARROLL, Mountain View, CA (US); Cory Yuen Fu MCLEAN, Newton, MA (US); Farhad I HORMOZDIARI, Watertown, MA (US)

(21) Appl. No.: 19/109,555

(22) PCT Filed: Aug. 4, 2023
(86) PCT No.: PCT/US2023/071648

§ 371 Date: Mar. 6, 2025

Related U.S. Application Data
(60) Provisional application No. 63/404,373, filed on Sep. 7, 2022.

(51) Int. Cl. G16B 40/20; G16B 25/10

(52) U.S. Cl. CPC G16B 40/20; G16B 25/10

(57) ABSTRACT

Improved methods for determining full-genome associations with phenotype data represented by medical images, ECG traces, spirometry-traces, or other high-dimensional phenotype-representing physiosignals are provided. These methods include training an encoder, as pan of an autoencoder, to project input physiosignals into a phenotypically representative set of lower-dimensional latent variables. In some examples, the latent variables are augmented by clinical correlates of the input physiosignals (e.g., a. force vital capacity-determined from a spirometry trace), The latent variables and/or clinical correlates are then used to determine genetic loci that are associated with each of the latent variables. These associations can then be used to focus drug development and/or to predict polygenic scores tor ram diseases for which sufficient, data, may-not be available for a full genome-wide association study or other genomic data-to-phenotype association.

### CROSS-REFERENCE TO RELATED APPLICATION

This application claims priority to U.S. Provisional Patent Application No. 63/404,373, filed on Sep. 7, 2022, the contents of which are hereby incorporated by reference in their entirety.

### BACKGROUND

Drug discovery or other biological investigations (e.g., the elucidation of the underlying cellular and/or genetic mechanisms of a disease state) can be facilitated by using genomic data (e.g., genomic sequence data, single nucleotide polymorphism (SNP) maps) and phenotype data (e.g., diagnosis with various conditions or diseases) in order to determine associations between the phenotype and the genomic data (e.g., a genome-wide association study (GWAS)). The results of such analyses can indicate which loci within the genome are most related to the phenotype of interest. The genes at such loci (e.g., regulatory genes, genes coding for proteins or other products) can then be determined, and drug candidates associated by the genes (e.g., drug candidates known or suspected to have effects on a product of the gene, drug candidates known or suspected to affect a regulatory network of which the gene is a part) could then be targeted for further assessment in order to develop treatments for diseases and/or conditions represented by the phenotype data analyzed.

The result of GWAS or other analyses of the associations between a phenotype and genomic data depends heavily on the sample size. However, collecting a large number of well-conditioned phenotype data (both with respect to noise/accuracy and distribution) and paired genomic data is often extremely difficult. Additionally, performing such analyses is computationally expensive (in terms of cycles, memory, storage, etc.). Accordingly, such studies are generally performed using simple, low-dimensional phenotype data, e.g., one-dimensional labels or measures related to a specific phenotype of interest (e.g., diagnosis with a specific disease or other condition). However, high-dimensional phenotype data (e.g., MRI or chest X-rays or other medical imaging data, ECG traces, etc.) is often available. It could be beneficial to perform GWAS or other genomic data-to-phenotype association analyses using such data directly, however, issues with the computational cost and statistical requirements for such data have prevented such developments.

### SUMMARY

In a first aspect, a method is provided that includes: (i) obtaining a first training dataset, wherein the first training dataset comprises a plurality of training examples, and wherein a given training example of the first training dataset includes one or more physiosignals; (ii) training an autoencoder using the training dataset to predict the physiosignals of the training examples of the first training dataset, wherein the autoencoder comprises an encoder that receives as an input one or more physiosignals of an input training example and outputs one or more latent variables, wherein the autoencoder also comprises a decoder that receives as an input the one or more latent variables from the encoder and outputs a prediction of the one or more physiosignals of the input training example; (iii) obtaining a second training dataset, wherein the second training dataset comprises a plurality of training examples, and wherein a given training example of the second training dataset includes one or more physiosignals and genomic data associated therewith; (iv) generating, for each training example of the second training dataset, a respective set of one or more latent variables, wherein generating a set of one or more latent variables for a particular training example of the second training dataset comprises applying the set of one or more physiosignals for the particular training example to the encoder of the trained autoencoder; and (v) determining a respective set of associations between each of the one or more latent variables and the genomic data of the second training dataset.

In a second aspect, a method is provided that includes: (i) obtaining an encoder that receives as an input one or more physiosignals and outputs one or more latent variables, wherein the encoder has been trained, as part of an autoencoder that also includes a decoder that receives as an input the one or more latent variables from the encoder and outputs one or more predicted physiosignals, to generate the one or more latent variables such that the decoder can predict the one or more physiosignals input into the encoder; (ii) obtaining a training dataset, wherein the training dataset comprises a plurality of training examples, and wherein a given training example of the training dataset includes one or more physiosignals and genomic data associated therewith; (iii) generating, for each training example of the training dataset, a respective set of one or more latent variables, wherein generating a set of one or more latent variables for a particular training example of the training dataset comprises applying the set of one or more physiosignals for the particular training example to the encoder; and (iv) determining a respective set of associations between each of the one or more latent variables and the genomic data of the training dataset.

In a third aspect, method is provided that includes: (i) obtaining a first training dataset, wherein the first training dataset comprises a plurality of training examples, and wherein a given training example of the first training dataset includes one or more physiosignals and one or more clinical correlates of the one or more physiosignals; (ii) training an autoencoder using the training dataset to predict the physiosignals of the training examples of the first training dataset, wherein the autoencoder comprises an encoder that receives as an input one or more physiosignals of an input training example and outputs one or more latent variables, wherein the autoencoder also comprises a decoder that receives as an input (a) the one or more latent variables from the encoder and (b) one or more clinical correlates of the input training example and outputs a prediction of the one or more physiosignals of the input training example; (iii) obtaining a second training dataset, wherein the second training dataset comprises a plurality of training examples, and wherein a given training example of the second training dataset includes one or more physiosignals, one or more clinical correlates of the one or more physiosignals, and genomic data associated therewith; (iv) generating, for each training example of the second training dataset, a respective set of one or more latent variables, wherein generating a set of one or more latent variables for a particular training example of the second training dataset comprises applying the set of one or more physiosignals for the particular training example to the encoder of the trained autoencoder; and (v) determining a respective set of associations between each of the one or more latent variables and the genomic data of the second training dataset.

In a fourth aspect, a non-transitory computer readable medium is provided having stored therein instructions executable by a computing device to cause the computing device to perform the method of the first, second, or third aspects.

In a fifth aspect, a system is provided that includes: (i) a controller comprising one or more processors; and (ii) a non-transitory computer readable medium having stored therein instructions executable by the controller device to cause the one or more processors to perform the method of the first, second, or third aspects.

The foregoing summary is illustrative only and is not intended to be in any way limiting. In addition to the illustrative aspects, embodiments, and features described above, further aspects, embodiments, and features will become apparent by reference to the figures and the following detailed description and the accompanying drawings.

### BRIEF DESCRIPTION OF THE FIGURES

FIG. 1A illustrates aspects of method for training a machine learning model, according to example embodiments.

FIG. 1B illustrates aspects of a method for using a trained machine learning model to improve the determination of genetic associations with phenotype information, according to example embodiments.

FIG. 1C illustrates aspects of method for training a machine learning model, according to example embodiments.

FIG. 1D illustrates aspects of a method for using known genetic associations with phenotype information to predict a genetic association with an additional phenotype, according to example embodiments.

FIG. 2 illustrates aspects of an example system.

FIG. 3 and FIG. 4 illustrate flowcharts of example methods.

FIG. 5 illustrates a flowchart of an example method.

FIG. 6 illustrates aspects of an example system.

FIG. 7A illustrates experimental data.

FIG. 7B illustrates experimental data.

FIG. 7C illustrates experimental data.

FIG. 7D illustrates experimental data.

FIG. 8 illustrates experimental data.

FIG. 9 illustrates experimental data.

FIG. 10 illustrates experimental data.

FIG. 11 illustrates experimental data.

FIG. 12 illustrates experimental data.

FIG. 13 illustrates experimental data.

FIG. 14 illustrates experimental data.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

### I. Overview

Genome-wide association studies (GWAS) or other techniques are used to determine the association between phenotype information (e.g., height, weight, blood type, diagnosis with a disease or condition, etc.) and genomic information (e.g., full chromosomal or full-genome sequences, SNP and/or variant maps or other information about the contents or condition of plurality of specific loci within the genome). Such techniques can be used to determine the overall level of heritability (the proportion of phenotypic variance explained by genetic data) of a phenotype and to determine what locations, or ranges of locations, within the genome are particularly genetically determinative of or otherwise associated with a phenotype of interest.

Such associational maps or other information can be applied in a variety of ways. In some examples, such maps can be used to determine an overall level of heritability of a phenotype (e.g., to determine a polygenic score (PGS) for the phenotype) which can then be used, e.g., to determine whether additional testing or other medical intervention is warranted to detect or manage the phenotype (e.g., to screen for susceptibility to a type of cancer or other disease or condition). In some examples, a map of the association of the phenotype with various loci within the genomic information (e.g., with a set of detected SNPs, base pairs, alleles, or other localized features within a genome) could be used to target drug development. For example, it could be determined that a particular disease or condition is associated with a set of loci within the genome, and drug targets related to those loci (e.g., that are known to act on a product of a gene at the identified loci, that are known to interact with a regulatory network involving genes or other features at the identified loci) could then be selected for further assessment as a potential treatment for the disease or condition.

Such associations can also be used to diagnose and/or determine the eventual likelihood of development of a disease or condition. For example, if certain SNPs, alleles, combinations or patterns of genomic features, or other genomic patterns are determined to be associated with a particular disease or condition (e.g., the development of breast cancer), genetic tests can be developed to detect such patterns. Individuals determined to have such a susceptibility can then receive further testing for diagnosis, could be provided with preventive or prophylactic treatment (e.g., prophylactic mastectomy), could engage in a schedule of enhanced screening to detect early the development of an associated disease or condition, could engage in lifestyle changes, or other treatments, diagnostics, or other actions.

As briefly outlined above, such phenotype-to-genomic information association analyses (e.g., GWAS) can provide significant benefits for the diagnosis and treatment of heritable diseases or conditions and/or diseases or conditions for which susceptibility thereto is heritable. However, the performance of such associational analyses require significant input data (large amounts of high-quality genomic and phenotypic data) and computational resources (e.g., computational cycles, memory, storage). Thus, such associational analyses have historically been limited to diseases or conditions that are represented relatively simply, e.g., by a single variable (e.g., a continuous variable, or a class label indicating “affected by condition of interest” or “control”) that is easily and unambiguously measured (e.g., a diagnosis with a disease or condition).

Higher-dimensional physiosignals, like medical imaging data (e.g., chest X-rays, MR images, CT scans), ECG traces, EEG traces, spirometry traces, or other physiosignals represent significant phenotype information. It would be beneficial to use such high-dimensional phenotype information to inform a genomic association study. This can involve determining, from the physiosignal information, specified clinical correlates (e.g., the density of tissue in specified anatomical regions of an MR image, a Q-R delay of an ECG, a forced vital capacity (FVC) of a spirometry trace) and then using those clinical correlates as the low-dimensional (e.g., one-dimensional) phenotype information applied for genomic association. However, such methods under-utilize significant phenotypic information represented by the complete high-dimensional image, trace, or other physiosignal data and may require clinical domain expertise to determine which clinical correlates are the most relevant. While such high-dimensional physiosignal data could be applied directly for genomic association (e.g., the intensity value of every pixel of an MR image, the voltage of every sample of an ECG trace, the flow rate or cumulative volume of every sample of a spirometry trace), such data is often noisy, and difficult to ‘clean’ prior to such application. Additionally, performance of genomic association studies on such high-dimensional data (e.g., hundreds or thousands of time-point samples of an ECG, EEG, or spirometry trace, thousands or millions of pixels of an MR, X-ray, or other medical image) is extremely computationally expensive, often ignores the correlation structure of such high-dimensional data (e.g., two consecutive time points of an ECG or two neighboring pixels of a medical image are highly correlated), and can lead to weaker statistical conclusions due to, e.g., excessive numbers of applied test (e.g., multiple-hypothesis corrections).

The embodiments described herein provide improvements on these limitations by using an autoencoder to discover embeddings of input physiosignals into a lower-dimensional space. A vector of ‘latent variables’ determined by mapping one or more physiosignals (e.g., spirometry traces) into the representative lower-dimensional space using the methods described herein has the benefit of representing the ‘generic function’ of the body system(s) (e.g., lungs and/or respiratory system) represented by the phenotype information present in the input physiosignals. Such lower-dimensional latent variable vectors thus represent more of the total phenotypic information represented in the input physiosignals while still being computationally tractable to apply to a genomic association analysis (rather than the hundreds, thousands, or millions of variables represented by the raw input physiosignal(s)). Such latent variables, representing as they do the ‘generic’ function of the relevant bodily system rather than any specific disease or condition, can also facilitate the determination of genomic-data-related information for novel or rare diseases for which insufficient data is available to perform GWAS or other genomic association analyses directly.

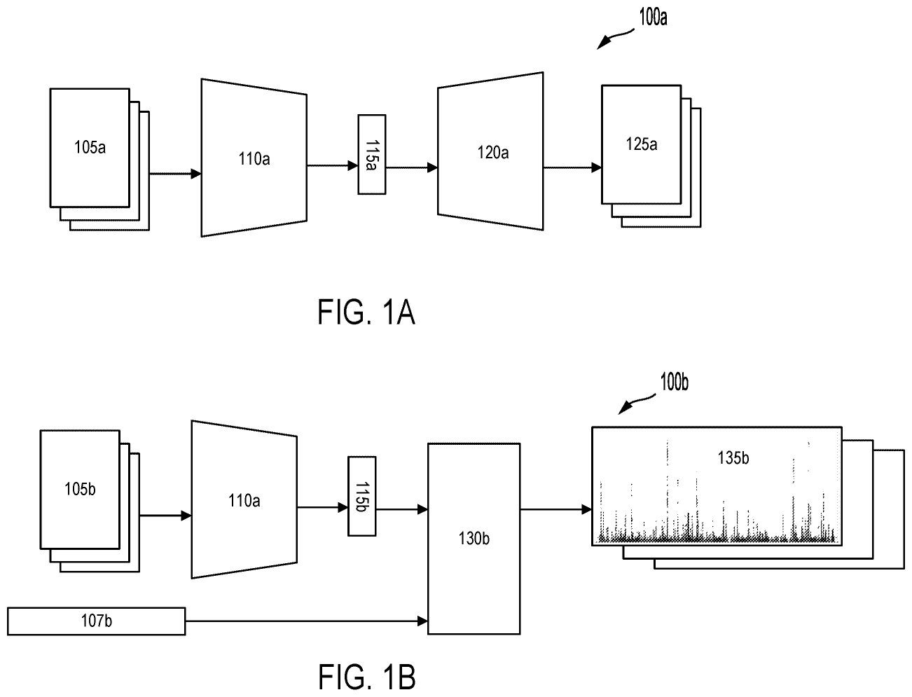

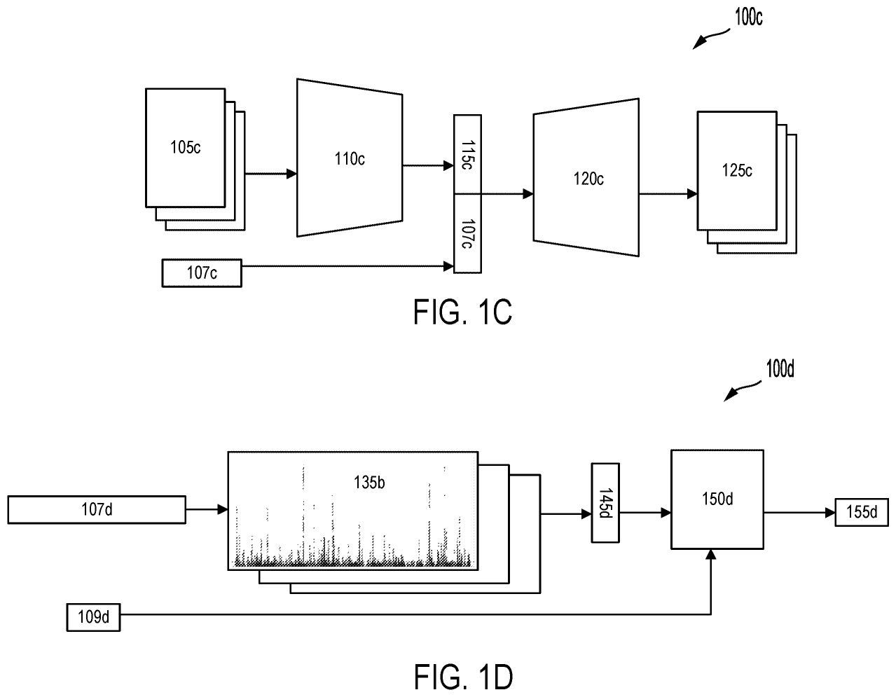

The embodiments described herein include training an autoencoder to replicate a set of training physiosignals (e.g., spirometry traces) via a low-dimensional (e.g., two-dimensional, five-dimensional) vector of latent variables. This is illustrated by way of example in FIG. 1A, which shows aspects of a process 100a for applying a particular example set of one or more physiosignals 105a (e.g., three spirometry traces representing a spirometric flow rate overtime of an expiration, a spirometric total volume expired over time of the expiration, and a spirometric flow rate normalized to total volume expired over time of an inspiration) to an autoencoder to replicate the example set of physiosignals 105a as an output set of one or more predicted physiosignals 125a. The autoencoder includes an encoder 110a that receives as an input the example set of physiosignals 105a and outputs a vector of one or more latent variables 115a. The autoencoder also includes a decoder 120a that receives the vector of one or more latent variables 115a as an input and outputs a predicted set of one or more physiosignals 125a (e.g., three spirometry traces). Differences between the input 115a and output 125a sets of one or more physiosignals can be determined (e.g., least-squares differences, some other cost function) for a plurality of example sets of physiosignals and used to update the encoder 110a and/or decoder 120a of the autoencoder such that the trained autoencoder is able to accurately replicate an input set of physiosignals as an output, and further such that the vector of one or more latent variables comes to represent phenotypic variation and structure in the set of input physiosignals.

The number of latent variables output from the encoder 110a can be selected such that the autoencoder is able to accurately replicate input physiosignals while also reducing the total number of latent variables in order to, e.g., reduce the computational cost of performing subsequent genomic association analyses. For example, where the set of physiosignals were three spirometry traces representing a spirometric flow rate over time of an expiration, a spirometric total volume expired over time of the expiration, and a spirometric flow rate normalized to total volume expired over time of an inspiration, a latent variable vector including five latent variables was found to permit accurate reconstruction of input sets of such spirometric traces.

The encoder 110a and decoder 120a could have a variety of model structures. In some examples, the encoder 110a could include a number of convolution and pooling filter layers (e.g., configured as a convolutional neural network), one for each input physiosignal, whose outputs are then presented to a multi-layer perceptron whose outputs are the latent variables 115a. In some examples, the decoder 120a could include a multi-layer perceptron that receives the input latent variables and whose outputs are presented, as inputs, to a number of transpose convolution and upsampling layers, one for each output physiosignal, whose outputs are the individual predicted output physiosignals 125a.

The encoder 110a and/or decoder 120a could be trained in a variety of different ways. For example, the autoencoder could be a variational autoencoder (VAE), with the outputs of the encoder 110a representing distributions for each of the latent variables (e.g., mean and variance of Gaussian distributions for each latent variable). Such a training method allows the training to account for noise in the training data and also to result in latent variables that are more disentangled and/or independent, thus representing more separable biological factors for later genomic association analyses. Such benefits can be enhanced by configuring and/or training the autoencoder as a beta(β)-variational autoencoder (β-VAE).

Once the encoder 110a has been trained as described above, it can be used to map input physiosignal(s) into the lower-dimensional, but still phenotypically representative, latent variable space for use in genomic association analyses (e.g., GWAS). This can include using the same set of training data used to train the encoder (e.g., the same sets of spirometric traces), if genomic data is available therefor, using a different set of training data, and/or using partially or fully overlapping sets of training data for the genomic association as for training the encoder 110a (e.g., training the encoder using a combination of a first dataset for which genomic data is available for the sets of physiosignal data and a second dataset that only includes sets of physiosignal data).

FIG. 1B illustrates aspects of an example process 100b for using an encoder 110a trained as described above to perform a genomic association analysis. An input set of physiosignals 105b for which genomic data 107b is also available (e.g., a sequence of one or more chromosome, an SNP map, a list or map of alleles or other known genetic variants, etc.) is applied to the encoder 110a to generate an embedding vector of one or more latent variables 115b. This is performed for a plurality of sets of physiosignals, and the resulting sets of latent variables are applied, along with their associated genomic information, to a genomic association analysis 130b (e.g., GWAS). The analysis 130b outputs a set of associations 135b between each of the one or more latent variables 115b and the genomic data. Thus, each association in the set of associations represents those loci of the genomic data that were associated with the corresponding latent variable.

In practice, many physiosignals are associated with corresponding traditional clinical correlates, which may in some examples be determined therefrom. For example, spirometry traces (e.g., flow rate over time of an expiration, total volume expired over time of the expiration, and flow rate normalized to total volume expired over time of an inspiration) are often used to determine a variety of clinical correlates, including but not limited to a Forced expiratory volume in 1 second (FEV1), a Forced vital capacity (FVC), a peak expiratory flow (PEF), a ratio of FEV1 over FVC (FEV1/FVC), a forced expiratory flow (FEF), a forced expiratory flow between specified fractions of expiration of the forced vital capacity (e.g., FEF25-75%), or some other clinical correlate of the spirometric trace(s). The methods described herein can be augmented by using a concatenation of a vector of such clinical correlates with the vector of latent variable for training of the autoencoder (i.e., for input to the decoder of the autoencoder) and/or for use in genomic association analyses. Such augmentation has a variety of benefits, including facilitation of clinical interpretation of the resulting genomic associations, reduction of the computational cost and amount of training data necessary to train the autoencoder (by allowing the length of the latent variable vector to be reduced, as some part of the phenotypic data in the input physiosignal data is already represented by the clinical correlates). Such augmentation also has the benefit of enriching the space spanned by the latent vectors, as they can now be trained to represent biological factors not already anticipated by the human-interpretable clinical correlates.

This is illustrated by way of example in FIG. 1C, which shows aspects of a process 100c for applying a particular example set of one or more physiosignals 105c (e.g., three spirometry traces representing a spirometric flow rate over time of an expiration, a spirometric total volume expired overtime of the expiration, and a spirometric flow rate normalized to total volume expired over time of an inspiration) to an autoencoder to replicate the example set of physiosignals 105c as an output set of one or more predicted physiosignals 125c. The autoencoder includes an encoder 110c that receives as an input the example set of physiosignals 105c and outputs a vector of one or more latent variables 115c. The autoencoder also includes a decoder 120c that receives, as an input, the vector of one or more latent variables 115c concatenated with a vector of one or more clinical correlates 107c, which may be determined from the set of physiosignals 105c. The decoder 120c outputs a predicted set of one or more physiosignals 125a. Differences between the input 115c and output 125c sets of one or more physiosignals can be determined (e.g., least-squares differences, some other cost function) for a plurality of example sets of physiosignals and used to update the encoder 110c and/or decoder 120c of the autoencoder such that the trained autoencoder is able to accurately replicate an input set of physiosignals as an output, and further such that the vector of one or more latent variables comes to represent phenotypic variation and structure in the set of input physiosignals that is not already represented by the clinical correlates 107c.

The number of latent variables output from the encoder 110c can be selected such that the autoencoder is able to accurately replicate input physiosignals while also reducing the total number of latent variables in order to, e.g., reduce the computational cost of performing subsequent genomic association analyses. For example, where the (i) set of physiosignals were three spirometry traces representing a spirometric flow rate over time of an expiration, a spirometric total volume expired over time of the expiration, and a spirometric flow rate normalized to total volume expired over time of an inspiration and (ii) the set of clinical correlates includes an FEV1, an FVC, a PEF, an FEV1/FVC, or an FEF25-75%, a latent variable vector including two latent variables was found to permit accurate reconstruction of input sets of such spirometric traces.

As noted above, one of the benefits of such methods is that the latent variables generated in such a manner are related generally to the overall generic function of the organ or system measured by the input physiosignals (e.g., lungs and/or respiratory system for spirometry), thus potentially leading to the discovery or more genomic loci that are relevant to the overall function of such organs or systems than if analyzing only phenotypic information related to specific diseases or conditions. This can lead to the identification of a broader range of candidate drugs or other potential treatments for additional investigation and/or assessment as part of a diagnostic. Such drug candidates can then be assessed with respect to specific maladies (e.g., chronic obstructive pulmonary disease, emphysema, etc. where the input physiosignals are related to lung function) and, where found efficacious, then provided to patients to treat such specific maladies.

Another benefit of such “generic function” latent variables is that they can be used to ‘bootstrap’ the determination of polygenic scores or other measures of heritable aspects of a phenotype for novel or rare conditions or other conditions for which insufficient data is available to perform a complete GWAS or other phenotype-to-genotype analysis. This can be accomplished by using the sets of associations for each of the latent variables (or for each of the latent variables and each of the clinical correlates, where the latent variables were augmented with clinical correlates to train the encoder) to determine a set of respective polygenic scores for genomic information (e.g., an SNP map) of a patient. These polygenic scores, which represent the estimated effect of the set of the patient's genetic variants (as represented in the genomic information) on each of the latent variables and/or clinical correlates, thus represent the patient's overall genetic risk or predisposition with respect to each of the aspects of “generic function” represented by the latent variables and/or clinical correlates (e.g., generic respiratory function, if the input physiosignals and/or clinical correlates thereof were spirometry traces or other respiratory-related physiosignals). These “generic” polygenic scores can then be applied to a model trained and/or fitted to predict a polygenic score or other output representative of the effect of the patient's genetic variants on a specific disease or condition (e.g., chronic obstructive pulmonary disease (COPD), asthma, or some other respiratory-related condition if the latent variables and/or clinical correlates are related to respiratory function). Because such a model can have significantly fewer parameters than the output of a GWAS or other genomic association analysis, the model can be trained/fitted with significantly fewer examples than would be needed for a GWAS or other genomic association analysis.

This is illustrated by way of example in FIG. 1D, which shows aspects of a process 100d that includes using sets of genetic associations 135b between each one of a set of one or more latent variables and/or clinical correlates as described herein to determine, for input genomic information of a patent 107d, respective polygenic scores 145d (or similar measures of overall genetic risk or predisposition) with respect to each of the latent variables and/or clinical correlates. These polygenic scores 145d are then applied to a trained and/or fitted model 150d to determine a polygenic score 155d or other output representative of the effect of the patient's genetic variants on a specific disease or condition for which the model 150d has been fitted and/or trained.

The model 150d can be fitted/trained in a variety of ways. For example, a plurality of sets of genomic information 107d and associated label information 109d for the specific disease or condition of interest could be obtained for a plurality of individuals. The genetic associations 135b could then be used, as described above, to determine, for each of the individuals and based on their respective genomic information 107d, a respective vector of one or more polygenic scores 145d (or similar measures of overall genetic risk or predisposition). These vectors, along with the associated label information 109d (e.g., indications of whether a particular individual is or is not diagnosed with the particular disease or condition of interest) could then be used to train and/or fit the model 150d. In some examples, the model could be a multi-layer perceptron or some other machine learning model. In some examples, the model could be a set of linear weights used to determine a linear combination of the polygenic scores 145d as the output polygenic score 155d.

Such polygenic scores determined via the method above can be used to diagnose and/or determine the eventual likelihood of development of the specific disease or condition, despite there having been insufficient information to perform a full GWAS or other genomic association analysis with respect to the specific condition. Such trained/fitted models can thus be used to develop genetic tests (by developing genetic tests for the underlying genetic patterns of association for each of the underlying latent variables and/or clinical correlated) to detect susceptibility to and/or likelihood to develop the specific disease or condition. Individuals determined to have such a susceptibility or likelihood can then receive further testing for diagnosis, could be provided with preventive or prophylactic treatment (e.g., prophylactic mastectomy), could engage in a schedule of enhanced screening to detect early the development of an associated disease or condition, could engage in lifestyle changes, or other treatments, diagnostics, or other actions.

### II. Illustrative Systems

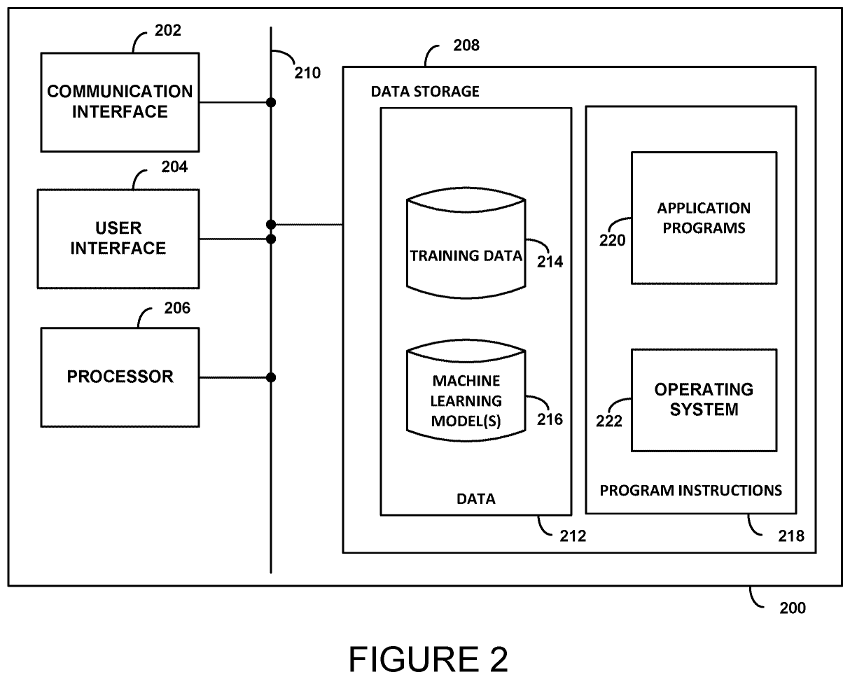

FIG. 2 illustrates an example system 200 that may be used to implement the methods described herein. By way of example and without limitation, system 200 may be a computer (such as a desktop, notebook, tablet, or handheld computer, a server), elements of a cloud computing system, or some other type of device. It should be understood that system 200 may represent a physical computing device such as a server, a particular physical hardware platform on which an physiosignal acquisition, genome data acquisition, machine learning, and/or other application operates in software, or other combinations of hardware and software that are configured to carry out functions as described herein. The system 200 could be a central system (e.g., a server, elements of a cloud computing system) that is configured to receive genomic information (e.g., a complete genome sequence, a map of SNPs, etc.), physiosignal information (e.g., spirometry traces, medical images), or other information from a remote system.

As shown in FIG. 2, system 200 may include a communication interface 202, a user interface 204, a processor 206, and data storage 208, all of which may be communicatively linked together by a system bus, network, or other connection mechanism 210.

Communication interface 202 may function to allow system 200 to communicate, using analog or digital modulation of electric, magnetic, electromagnetic, optical, or other signals, with other devices, access networks, and/or transport networks. Thus, communication interface 202 may facilitate circuit-switched and/or packet-switched communication, such as plain old telephone service (POTS) communication and/or Internet protocol (IP) or other packetized communication. For instance, communication interface 202 may include a chipset and antenna arranged for wireless communication with a radio access network or an access point. Also, communication interface 202 may take the form of or include a wireline interface, such as an Ethernet, Universal Serial Bus (USB), or High-Definition Multimedia Interface (HDMI) port. Communication interface 202 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 202. Furthermore, communication interface 202 may comprise multiple physical communication interfaces (e.g., a Wifi interface, a BLUETOOTH® interface, and a wide-area wireless interface). In some embodiments, communication interface 202 may function to allow system 200 to communicate with other devices, remote servers, access networks, and/or transport networks.

User interface 204 may function to allow system 200 to interact with a user or other entity, for example to receive input from and/or to provide output to the user. Thus, user interface 204 may include input components such as a keypad, keyboard, touch-sensitive or presence-sensitive panel, computer mouse, trackball, joystick, microphone, and so on. User interface 104 may also include one or more output components such as a display screen which, for example, may be combined with a presence-sensitive panel. The display screen may be based on CRT, LCD, and/or LED technologies, or other technologies now known or later developed. User interface 204 may also be configured to generate audible output(s), via a speaker, speaker jack, audio output port, audio output device, earphones, and/or other similar devices.

Processor 206 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of performing genome-wide association studies or other analyses to determine associations between genomic information and phenotype information, executing rule-based and/or machine learning models, training machine learning models, among other applications or functions. Data storage 208 may include one or more volatile and/or non-volatile storage components, such as magnetic, optical, flash, or organic storage, and may be integrated in whole or in part with processor 206. Data storage 208 may include removable and/or non-removable components.

Processor 206 may be capable of executing program instructions 218 (e.g., compiled or non-compiled program logic and/or machine code) stored in data storage 208 to carry out the various functions described herein. Therefore, data storage 208 may include a non-transitory computer-readable medium, having stored thereon program instructions that, upon execution by system 200, cause system 200 to carry out any of the methods, processes, or functions disclosed in this specification and/or the accompanying drawings. The execution of program instructions 218 by processor 206 may result in processor 206 using data 212.

By way of example, program instructions 218 may include an operating system 222 (e.g., an operating system kernel, device driver(s), and/or other modules) and one or more application programs 220 (e.g., functions for executing and/or training a machine learning model, for performing a genome-wide association study or other genomic information-to-phenotype association) installed on system 200. Data 212 may include training data (e.g. sets of spirometry traces or other physiosignal data that can be used to train an autoencoder, clinical correlates of such physiosignal information that could be used to augment training of the autoencoder, genomic information that could be used to determine associations between the genomic data for individuals and corresponding latent variables determined for such individuals, etc.) 214 and/or machine learning model(s) 216 (e.g., trained encoders for receiving input physiosignal data and outputting latent variables representing such physiosignal data) that may be determined therefrom or obtained in some other manner.

Application programs 220 may communicate with operating system 222 through one or more application programming interfaces (APIs). These APIs may facilitate, for instance, application programs 220 transmitting or receiving information via communication interface 202, receiving and/or displaying information on user interface 204, and so on.

Application programs 220 may take the form of “apps” that could be downloadable to system 200 through one or more online application stores or application markets (via, e.g., the communication interface 202). However, application programs can also be installed on system 200 in other ways, such as via a web browser or through a physical interface (e.g., a USB port) of the system 200.

### III. Example Methods

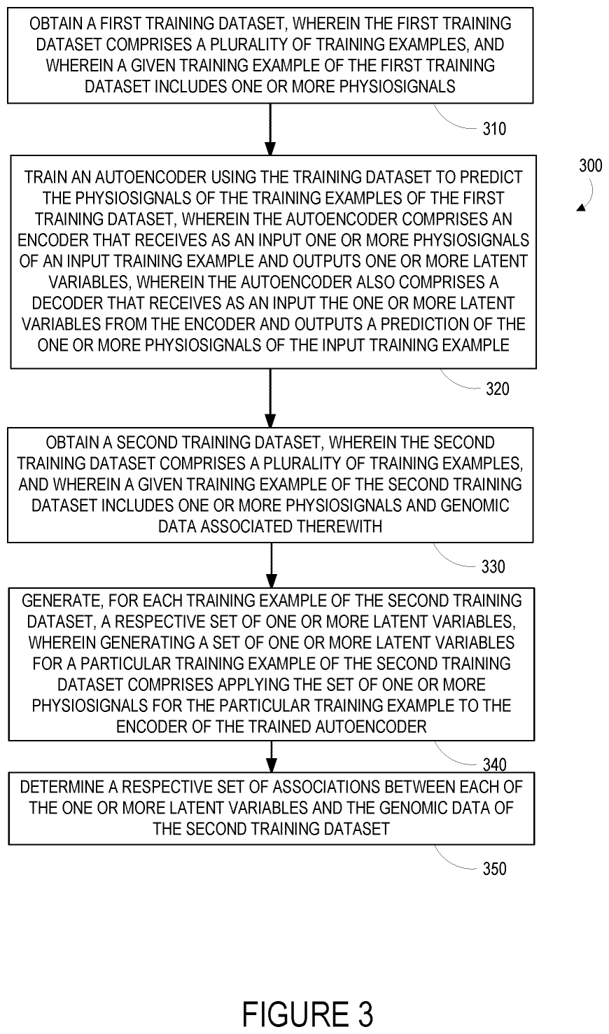

FIG. 3 is a flowchart of an example method 300. The method 300 includes obtaining a first training dataset, wherein the first training dataset comprises a plurality of training examples, and wherein a given training example of the first training dataset includes one or more physiosignals (310). The method 300 additionally includes training an autoencoder using the training dataset to predict the physiosignals of the training examples of the first training dataset, wherein the autoencoder comprises an encoder that receives as an input one or more physiosignals of an input training example and outputs one or more latent variables, wherein the autoencoder also comprises a decoder that receives as an input the one or more latent variables from the encoder and outputs a prediction of the one or more physiosignals of the input training example (320). The method 300 additionally obtaining a second training dataset, wherein the second training dataset comprises a plurality of training examples, and wherein a given training example of the second training dataset includes one or more physiosignals and genomic data associated therewith (330). The method 300 additionally includes generating, for each training example of the second training dataset, a respective set of one or more latent variables, wherein generating a set of one or more latent variables for a particular training example of the second training dataset comprises applying the set of one or more physiosignals for the particular training example to the encoder of the trained autoencoder (340). The method 300 additionally includes determining a respective set of associations between each of the one or more latent variables and the genomic data of the second training dataset (350). The method 300 could include additional or alternative features.

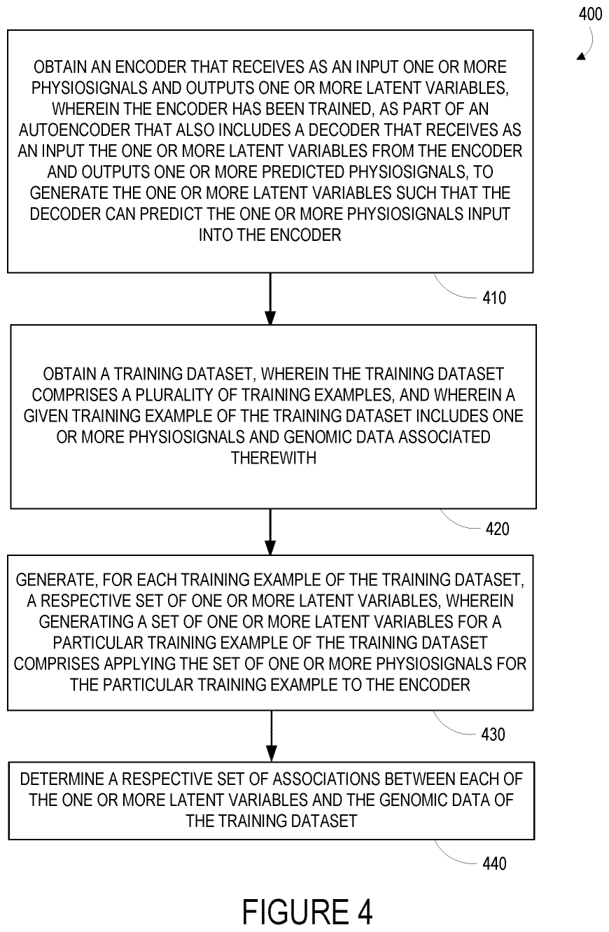

FIG. 4 is a flowchart of an example computer-implemented method 400. The method 400 includes obtaining an encoder that receives as an input one or more physiosignals and outputs one or more latent variables, wherein the encoder has been trained, as part of an autoencoder that also includes a decoder that receives as an input the one or more latent variables from the encoder and outputs one or more predicted physiosignals, to generate the one or more latent variables such that the decoder can predict the one or more physiosignals input into the encoder (410). The method 400 additionally obtaining a training dataset, wherein the training dataset comprises a plurality of training examples, and wherein a given training example of the training dataset includes one or more physiosignals and genomic data associated therewith (420). The method 400 additionally includes generating, for each training example of the training dataset, a respective set of one or more latent variables, wherein generating a set of one or more latent variables for a particular training example of the training dataset comprises applying the set of one or more physiosignals for the particular training example to the encoder (430). The method 400 also includes determining a respective set of associations between each of the one or more latent variables and the genomic data of the training dataset (440). The method 400 could include additional or alternative features.

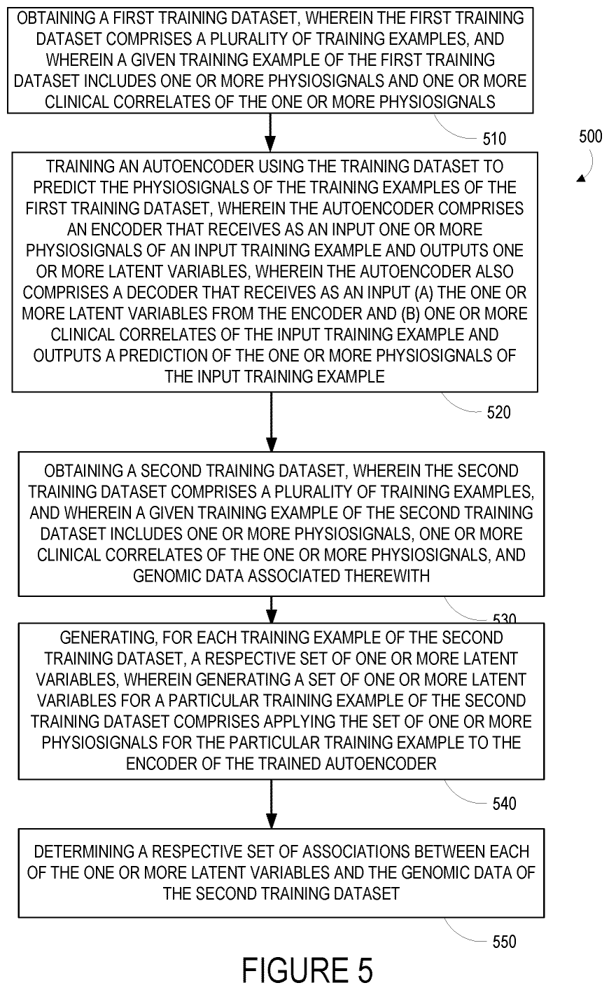

FIG. 5 is a flowchart of an example computer-implemented method 500. The method 500 includes obtaining a first training dataset, wherein the first training dataset comprises a plurality of training examples, and wherein a given training example of the first training dataset includes one or more physiosignals and one or more clinical correlates of the one or more physiosignals (510). The method 500 additionally includes training an autoencoder using the training dataset to predict the physiosignals of the training examples of the first training dataset, wherein the autoencoder comprises an encoder that receives as an input one or more physiosignals of an input training example and outputs one or more latent variables, wherein the autoencoder also comprises a decoder that receives as an input (i) the one or more latent variables from the encoder and (ii) one or more clinical correlates of the input training example and outputs a prediction of the one or more physiosignals of the input training example (520). The method 500 additionally includes obtaining a second training dataset, wherein the second training dataset comprises a plurality of training examples, and wherein a given training example of the second training dataset includes one or more physiosignals, one or more clinical correlates of the one or more physiosignals, and genomic data associated therewith (530). The method 500 additionally includes generating, for each training example of the second training dataset, a respective set of one or more latent variables, wherein generating a set of one or more latent variables for a particular training example of the second training dataset comprises applying the set of one or more physiosignals for the particular training example to the encoder of the trained autoencoder (540). The method 500 additionally includes determining a respective set of associations between each of the one or more latent variables and the genomic data of the second training dataset (550). The method 500 could include additional or alternative features.

### IV. Example Machine Learning Models and Training Thereof

A machine learning model as described herein may include, but is not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures.

An artificial neural network (ANN) could be configured in a variety of ways. For example, the ANN could include two or more layers, could include units having linear, logarithmic, or otherwise-specified output functions, could include fully or otherwise-connected neurons, could include recurrent and/or feed-forward connections between neurons in different layers, could include filters or other elements to process input information and/or information passing between layers, or could be configured in some other way to facilitate the generation of predicted color palettes based on input images.

An ANN could include one or more filters that could be applied to the input (or to the output of some intermediate layer of the ANN) and the outputs of such filters could then be applied to the inputs of one or more neurons of the ANN. For example, such an ANN could be or could include a convolutional neural network (CNN). Convolutional neural networks are a variety of ANNs that are configured to facilitate ANN-based classification or other processing based on images or other large-dimensional inputs whose elements are organized within two or more dimensions. The organization of the ANN along these dimensions may be related to some structure in the input (e.g., as relative location within the two-dimensional space of an image can be related to similarity between pixels of the image).

In example embodiments, a CNN includes at least one two-dimensional (or higher-dimensional) filter that is applied to an input; the filtered input is then applied to neurons of the CNN (e.g., of a convolutional layer of the CNN). The convolution of such a filter and an input could represent the color values of a pixel or a group of pixels from the input, in embodiments where the input is an image. A set of neurons of a CNN could receive respective inputs that are determined by applying the same filter to an input. Additionally or alternatively, a set of neurons of a CNN could be associated with respective different filters and could receive respective inputs that are determined by applying the respective filter to the input. Such filters could be trained during training of the CNN or could be pre-specified. For example, such filters could represent wavelet filters, center-surround filters, biologically-inspired filter kernels (e.g., from studies of animal visual processing receptive fields), or some other pre-specified filter patterns.

A CNN or other variety of ANN could include multiple convolutional layers (e.g., corresponding to respective different filters and/or features), pooling layers, rectification layers, fully connected layers, or other types of layers. Convolutional layers of a CNN represent convolution of an input image, or of some other input (e.g., of a filtered, downsampled, or otherwise-processed version of an input image), with a filter. Pooling layers of a CNN apply non-linear downsampling to higher layers of the CNN, e.g., by applying a maximum, average, L2-norm, or other pooling function to a subset of neurons, outputs, or other features of the higher layer(s) of the CNN. Rectification layers of a CNN apply a rectifying nonlinear function (e.g., a non-saturating activation function, a sigmoid function) to outputs of a higher layer. Fully connected layers of a CNN receive inputs from many or all of the neurons in one or more higher layers of the CNN. The outputs of neurons of one or more fully connected layers (e.g., a final layer of an ANN or CNN) could be used to determine information about areas of an input image (e.g., for each of the pixels of an input image) or for the image as a whole.

Neurons in a CNN can be organized according to corresponding dimensions of the input. For example, where the input is an image (a two-dimensional input, or a three-dimensional input where the color channels of the image are arranged along a third dimension), neurons of the CNN (e.g., of an input layer of the CNN, of a pooling layer of the CNN) could correspond to locations in the two-dimensional input image. Connections between neurons and/or filters in different layers of the CNN could be related to such locations. For example, a neuron in a convolutional layer of the CNN could receive an input that is based on a convolution of a filter with a portion of the input image, or with a portion of some other layer of the CNN, that is at a location proximate to the location of the convolutional-layer neuron. In another example, a neuron in a pooling layer of the CNN could receive inputs from neurons, in a layer higher than the pooling layer (e.g., in a convolutional layer, in a higher pooling layer), that have locations that are proximate to the location of the pooling-layer neuron.

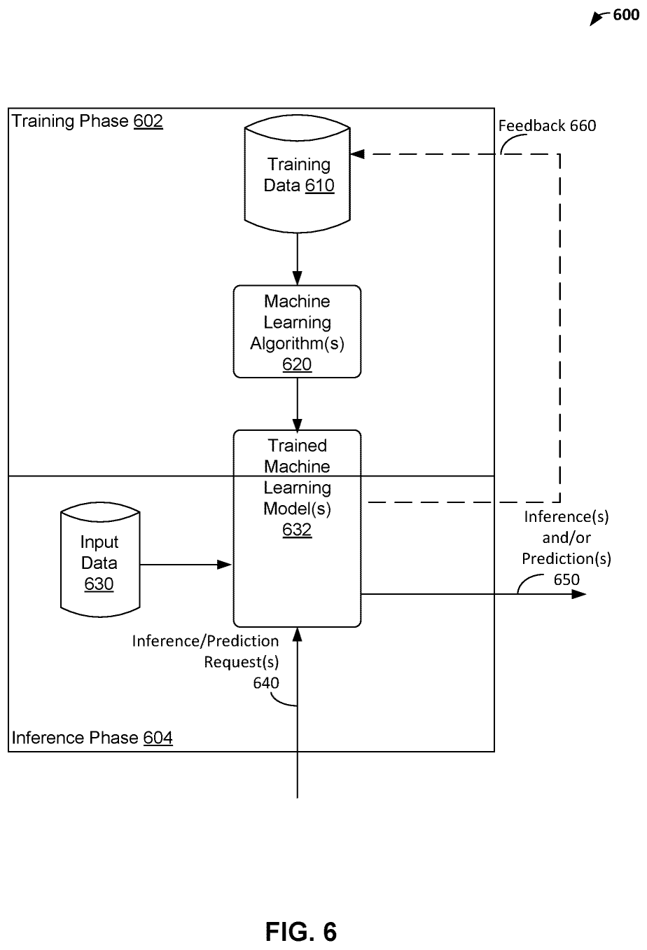

FIG. 6 shows diagram 600 illustrating a training phase 602 and an inference phase 604 of trained machine learning model(s) 632, in accordance with example embodiments. Some machine learning techniques involve training one or more machine learning algorithms, on an input set of training data to recognize patterns in the training data and provide output inferences and/or predictions about (patterns in the) training data. Such output could take the form of filtered or otherwise modified versions of the input, e.g., an input image could be a color-swapped version of an image of a cell, in order to prevent the model being trained from associating certain morphological features with specific color inputs. The resulting trained machine learning algorithm can be termed as a trained machine learning model. For example, FIG. 6 shows training phase 602 where one or more machine learning algorithms 620 are being trained on training data 610 to become trained machine learning model 632. Then, during inference phase 604, trained machine learning model 632 can receive input data 630 and one or more inference/prediction requests 640 (perhaps as part of input data 630) and responsively provide as an output one or more inferences and/or predictions 650.

As such, trained machine learning model(s) 632 can include one or more models of one or more machine learning algorithms 620. Machine learning algorithm(s) 620 may include, but are not limited to: an artificial neural network (e.g., herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures. Machine learning algorithm(s) 620 may be supervised or unsupervised, and may implement any suitable combination of online and offline learning.

In some examples, machine learning algorithm(s) 620 and/or trained machine learning model(s) 632 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine learning algorithm(s) 620 and/or trained machine learning model(s) 632. In some examples, trained machine learning model(s) 632 can be trained, reside and execute to provide inferences on a particular computing device, and/or otherwise can make inferences for the particular computing device.

During training phase 602, machine learning algorithm(s) 620 can be trained by providing at least training data 610 as training input using unsupervised (or “self-supervised”), supervised, semi-supervised, and/or reinforcement learning techniques. Unsupervised learning involves providing a portion (or all) of training data 610 to machine learning algorithm(s) 620 and machine learning algorithm(s) 620 determining one or more output inferences based on the provided portion (or all) of training data 610. Supervised learning involves providing a portion of training data 610 to machine learning algorithm(s) 620, with machine learning algorithm(s) 620 determining one or more output inferences based on the provided portion of training data 610, and the output inference(s) are either accepted or corrected based on correct results associated with training data 610. In some examples, supervised learning of machine learning algorithm(s) 620 can be governed by a set of rules and/or a set of labels for the training input, and the set of rules and/or set of labels may be used to correct inferences of machine learning algorithm(s) 620.

Semi-supervised learning involves having correct results for part, but not all, of training data 610. During semi-supervised learning, supervised learning is used for a portion of training data 610 having correct results, and unsupervised learning is used for a portion of training data 610 not having correct results. Reinforcement learning involves machine learning algorithm(s) 620 receiving a reward signal regarding a prior inference, where the reward signal can be a numerical value. During reinforcement learning, machine learning algorithm(s) 620 can output an inference and receive a reward signal in response, where machine learning algorithm(s) 620 are configured to try to maximize the numerical value of the reward signal. In some examples, reinforcement learning also utilizes a value function that provides a numerical value representing an expected total of the numerical values provided by the reward signal over time. In some examples, machine learning algorithm(s) 620 and/or trained machine learning model(s) 632 can be trained using other machine learning techniques, including but not limited to, incremental learning and curriculum learning.

In some examples, machine learning algorithm(s) 620 and/or trained machine learning model(s) 632 can use transfer learning techniques. For example, transfer learning techniques can involve trained machine learning model(s) 632 being pre-trained on one set of data and additionally trained using training data 610. More particularly, machine learning algorithm(s) 620 can be pre-trained on data from one or more computing devices and a resulting trained machine learning model provided to computing device CD1, where CD1 is intended to execute the trained machine learning model during inference phase 604. Then, during training phase 602, the pre-trained machine learning model can be additionally trained using training data 610, where training data 610 can be derived from kernel and non-kernel data of computing device CD1. This further training of the machine learning algorithm(s) 620 and/or the pre-trained machine learning model using training data 610 of CD1's data can be performed using either supervised or unsupervised learning. Once machine learning algorithm(s) 620 and/or the pre-trained machine learning model has been trained on at least training data 610, training phase 602 can be completed. The trained resulting machine learning model can be utilized as at least one of trained machine learning model(s) 632.

In particular, once training phase 602 has been completed, trained machine learning model(s) 632 can be provided to a computing device, if not already on the computing device. Inference phase 604 can begin after trained machine learning model(s) 432 are provided to computing device CD1.

During inference phase 604, trained machine learning model(s) 632 can receive input data 630 and generate and output one or more corresponding inferences and/or predictions 650 about input data 630. As such, input data 630 can be used as an input to trained machine learning model(s) 632 for providing corresponding inference(s) and/or prediction(s) 650 to kernel components and non-kernel components. For example, trained machine learning model(s) 632 can generate inference(s) and/or prediction(s) 650 in response to one or more inference/prediction requests 640. In some examples, trained machine learning model(s) 632 can be executed by a portion of other software. For example, trained machine learning model(s) 632 can be executed by an inference or prediction daemon to be readily available to provide inferences and/or predictions upon request. Input data 630 can include data from computing device CD1 executing trained machine learning model(s) 632 and/or input data from one or more computing devices other than CD1.

Input data 630 can include a collection of images provided by one or more sources. The collection of images can include video frames, images resident on computing device CD1, and/or other images. Other types of input data are possible as well.

Inference(s) and/or prediction(s) 650 can include output physiosignals (e.g., medical images, ECG traces, spirometry traces), output latent vectors that are embedded in a multi-dimensional space and that represent a projection of an input (e.g., one or more spirometry traces) into the multi-dimensional space, numerical values, polygenic scores, classifier outputs indicative of a phenotype, classifier outputs indicative of whether a disease state or other condition is indicated by input genomic data, and/or other output data produced by trained machine learning model(s) 632 operating on input data 630 (and training data 610). In some examples, trained machine learning model(s) 632 can use output inference(s) and/or prediction(s) 650 as input feedback 660. Trained machine learning model(s) 632 can also rely on past inferences as inputs for generating new inferences.

### V. Experimental Results

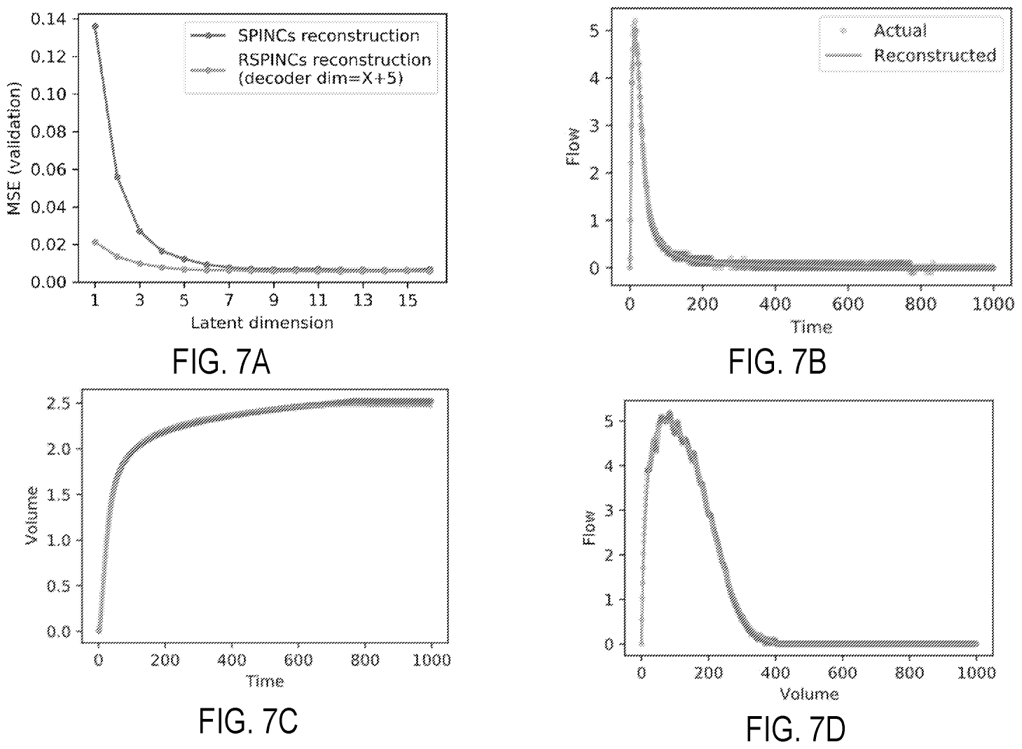

FIG. 7A illustrates the ability of the methods described herein to accurately replicate input sets of spirometric traces, measured with respect to mean squared error (MSE) in a validation set of spirometric traces. Results are shown for latent-variable-only models (“SPINCs reconstruction,” SPINC=spirogram encoding) and for latent-variable-plus-clinical correlates models (“RSPINCs reconstruction,” RSPINC=residual spirogram encoding, the set of clinical correlates was FEV1. FVC, PEF, FEV1/FVC, and FEF25-75%). FIGS. 7B-7D show examples of the input spirogram traces and the reconstructed traces generated by these methods.

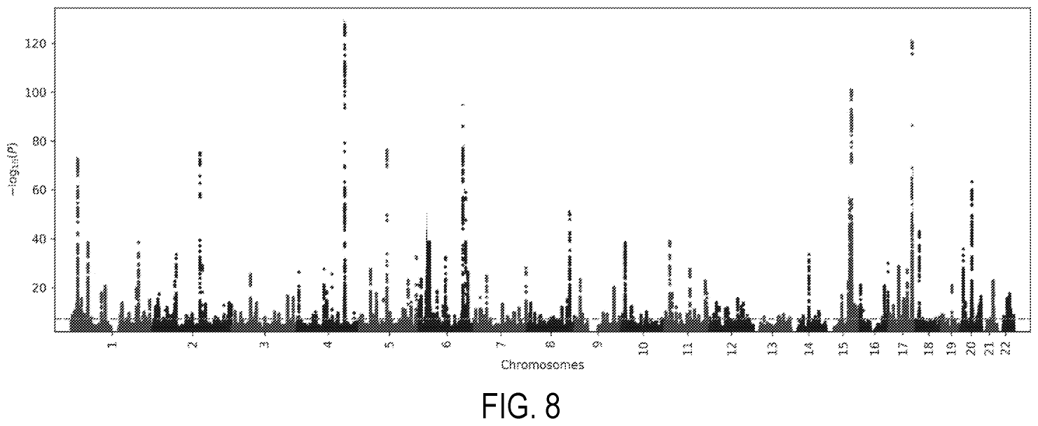

FIG. 8 depicts the results of a GWAS performed on two latent variables developed by training an autoencoder with FEV1, FVC, PEF, FEV1/FVC, and FEF25-75% to augment the latent variables. One of the latent variables had a heritability of 16.1%.

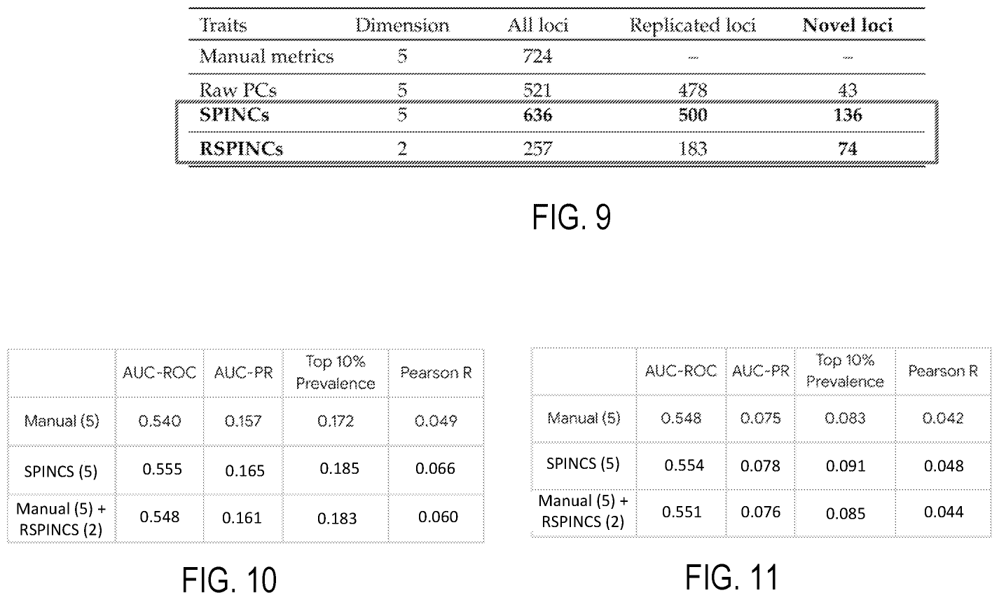

FIG. 9 depicts the number of genetic loci with super-threshold associations (“significant loci”) determined by GWAS against a variety of sets of variables (FEV1, FVC, PEF, FEV1/FVC, and FEF25-75% for “manual metrics,” a principal components decomposition of the raw spirogram traces for “Raw PCs,” a set of five latent variables for “SPINCs,” and a set of two latent variables and FEV1, FVC, PEF, FEV1/FVC, and FEF25-75% for “RSPINCs”). The loci identified by each of the methods, relative to the loci identified via the “manual metrics” method, are also reported as whether they were novel with respect to the “manual metrics” method, or simply identified loci also identified in the “manual metrics” method. The most significant loci identified via the methods described herein that were NOT previously discovered via GWAS for lung-related traits included the genes ALX4, ZNF703, WNT11, B4GALNT4, KLF12, BMP{3,7}, XRRA1, TRIOBP, SEMA6D, TMEM174, LINC01151, SOX6, PLEKHA6, and ZFPM2.

FIGS. 10 and 11 depict the ability of the methods described herein to develop useful ‘bootstrapped’ polygenic scores for asthma and COPD (as examples of “specific diseases or conditions”) based on the associations determined for the latent variables and/or clinical correlates as described herein. The ability of the ‘bootstrapped’ output scores (determined using a model consisting of linear weights of the input set of latent variable and/or clinical correlate polygenic scores) to predict the presence of asthma or COPD are represented by the area-under-the-curve of the receiver-operating-characteristic (AUC-ROC), the area-under-the-curve of the precision-recall curve (AUC-PR), the prevalence of COPD/asthma within the top 10% of individuals with respect to the predicted polygenic score, and by Pearson's R. These results are reported for both the five-latent-variables model (SPINCs) and the two-latent-variables plus five-clinical-correlates model (Manual+RSPINCs).

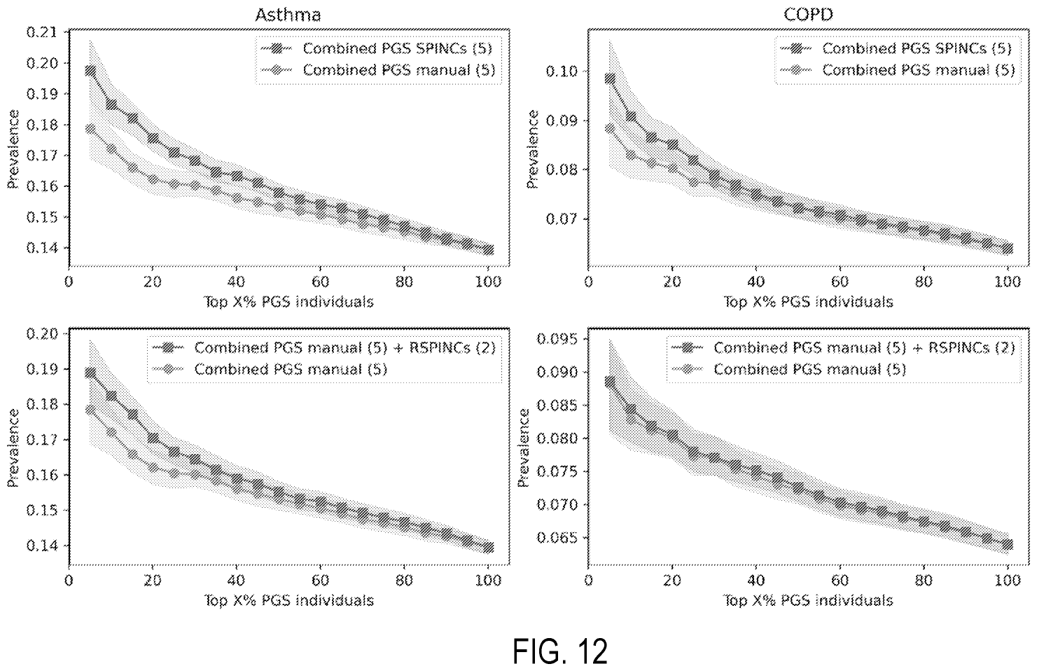

FIG. 12 depicts the prevalence of COPD or asthma within the top N % of individuals (with N having discrete values ranging from 5% to 100%) with respect to the predicted polygenic score, with the polygenic score predicted based on five manually-selected clinical correlates, a set of five latent variables, or a combination of the five manually-selected clinical correlates and two latent variables trained along with the manually-selected variable (e.g., as the RSPINCs above).

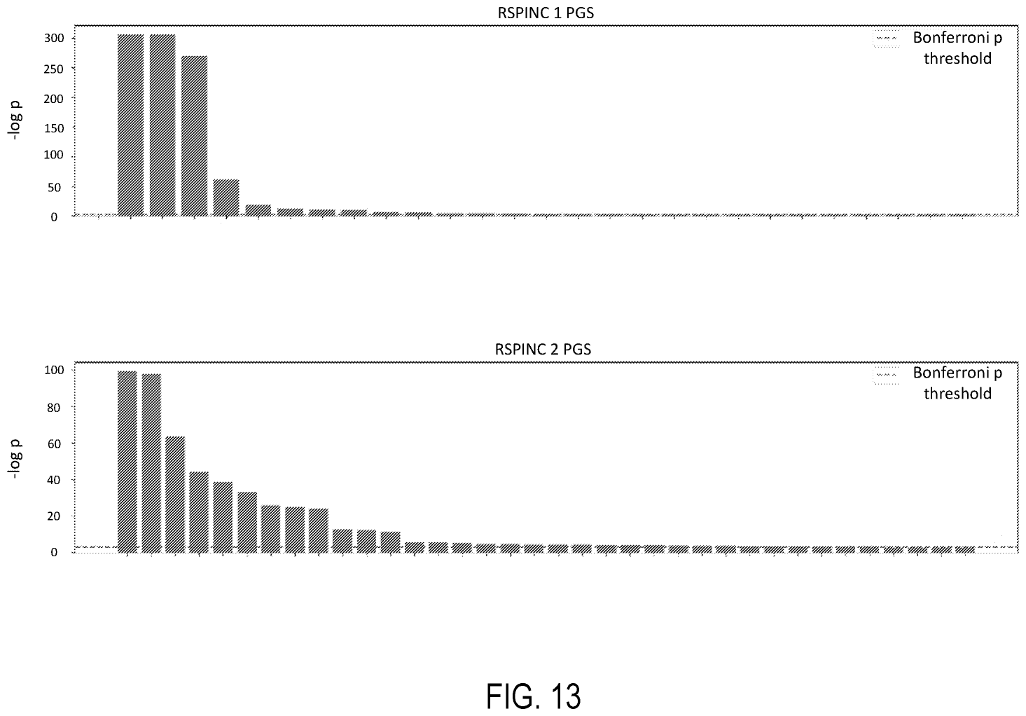

FIG. 13 depicts the ability (in terms of negative log probability) of each of the two latent variables (of the two-latent-variables plus five-clinical-correlates model) to predict a number of different clinical and/or phenotypic correlates for which data was available.

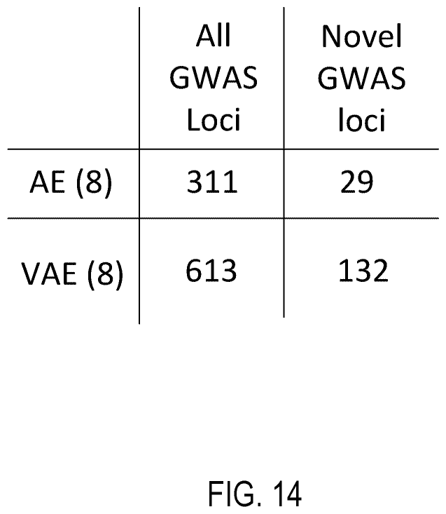

FIG. 14 depicts the ability of the methods described herein to generate latent variables that can be used to identify significant genetic loci, and in particular, significant genetic loci that are novel relative to prior GWAS studies. As shown, the use of a simple autoencoder (“AE”) to train the latent variables resulted in the identification of 311 significant loci, with 29 of those loci being novel. When a variational autoencoder (“VAE”) was instead used, the total number of identified significant loci increased to 613, with 132 of those loci being novel.

### VI. Conclusion

The particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

Additionally, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

1. A method comprising:
obtaining a first training dataset, wherein the first training dataset comprises a plurality of training examples, and wherein a given training example of the first training dataset includes one or more physiosignals;
training an autoencoder using the training dataset to predict the physiosignals of the training examples of the first training dataset, wherein the autoencoder comprises an encoder that receives as an input one or more physiosignals of an input training example and outputs one or more latent variables, wherein the autoencoder also comprises a decoder that receives as an input the one or more latent variables from the encoder and outputs a prediction of the one or more physiosignals of the input training example;
obtaining a second training dataset, wherein the second training dataset comprises a plurality of training examples, and wherein a given training example of the second training dataset includes one or more physiosignals and genomic data associated therewith;
generating, for each training example of the second training dataset, a respective set of one or more latent variables, wherein generating a set of one or more latent variables for a particular training example of the second training dataset comprises applying the set of one or more physiosignals for the particular training example to the encoder of the trained autoencoder; and
determining a respective set of associations between each of the one or more latent variables and the genomic data of the second training dataset.

2. The method of claim 1, wherein the autoencoder includes a variational autoencoder.

3. The method of claim 2, wherein the variational autoencoder is a β-variational autoencoder.

4. The method of claim 1, wherein the one or more physiosignals received by the encoder as an input comprise a spirometric flow rate over time of an expiration, a spirometric total volume expired over time of the expiration, and a spirometric flow rate normalized to total volume expired over time of an inspiration.

5. The method of claim 4, wherein the one or more latent variables output by the encoder consist of five latent variables.

6. The method of claim 1, wherein the decoder also receives as an input one or more clinical correlates of the one or more physiosignals received as an input by the encoder, and wherein the decoder outputs the prediction of the one or more physiosignals of the input training example based on the input one or more latent variables and one or more clinical correlates.

7. The method of claim 6, wherein the one or more physiosignals received by the encoder as an input comprise a spirometric flow rate over time of an expiration, a spirometric total volume expired over time of the expiration, and a spirometric flow rate normalized to total volume expired over time of an inspiration, and wherein the one or more clinical correlates of the one or more physiosignals comprise at least one of an FEV1, an FVC, a PEF, an FEV1/FVC, or an FEF25-75%.

8. The method of claim 7, wherein the one or more latent variables output by the encoder consist of two latent variables and wherein the he one or more clinical correlates of the one or more physiosignals comprise an FEV1, an FVC, a PEF, an FEV1/FVC, and an FEF25-75%.

9. The method of claim 1, further comprising:
based on the determined sets of associations between each of the one or more latent variables and the genomic data of the second training dataset, identifying at least one gene;
based on the identified at least one gene, selecting a drug candidate known to have a mechanism of action associated with the identified at least one gene;
assessing the selected drug candidate for clinical efficacy; and
based on the assessed clinical efficacy of the selected drug candidate, treating a patient using the selected drug candidate.

10. The method of claim 1, further comprising:
obtaining a third training dataset, wherein the third training dataset comprises a plurality of training examples, and wherein a given training example of the third training dataset includes genomic data and a phenotype label associated therewith that is indicative of whether the genomic data is from a person who has been diagnosed with a specified disease or condition;
determining, using the determined sets of associations between each of the one or more latent variables and the genomic data of the second training dataset, a set of polygenic scores for each training example in the third training dataset, wherein determining a set of polygenic scores for a particular training example in the third training dataset comprises determining a respective polygenic score for each of the one or more latent variables;
using the determined sets of polygenic scores and the phenotype labels of the third training dataset, training a model to predict a polygenic score with respect to the specified disease or condition based on a set of polygenic scores determined for the one or more latent variables;
obtaining additional genomic data for a patient;
based on the additional genomic data, determining a set of polygenic scores for the patient that includes a respective polygenic score for each of the one or more latent variables;
applying the set of polygenic scores determined for the patient to the model to generate a polygenic score for the patient with respect to the specified disease or condition; and
based on the generated polygenic score for the patient with respect to the specified disease or condition, prescribing a drug to the patient, providing a treatment to the patient, or performing a diagnostic on the patient.

11.-24. (canceled)

25. A method comprising:
obtaining an encoder that receives as an input one or more physiosignals and outputs one or more latent variables, wherein the encoder has been trained, as part of an autoencoder that also includes a decoder that receives as an input the one or more latent variables from the encoder and outputs one or more predicted physiosignals, to generate the one or more latent variables such that the decoder can predict the one or more physiosignals input into the encoder;
obtaining one or more physiosignals for a patient;
applying the obtained one or more physiosignals for the patient to the encoder to generate one or more latent variables for the patient; and
based on the generated one or more latent variables for the patient, prescribing a drug to the patient, providing a treatment to the patient, or performing a diagnostic on the patient.

26. The method of claim 25, wherein the one or more physiosignals comprise a spirometric flow rate over time of an expiration, a spirometric total volume expired over time of the expiration, and a spirometric flow rate normalized to total volume expired over time of an inspiration.

27. The method of claim 26, wherein the one or more latent variables output by the encoder consist of five latent variables.

28. The method of claim 25, wherein prescribing a drug to the patient, providing a treatment to the patient, or performing a diagnostic on the patient based on the generated one or more latent variables for the patient comprises prescribing a drug to the patient, providing a treatment to the patient, or performing a diagnostic on the patient based on the generated one or more latent variables for the patient and based on one or more clinical correlates of the one or more physiosignals for the patient, wherein the encoder has been trained, as part of the autoencoder that also includes the decoder that receives as an input the one or more latent variables from the encoder and that also receives as input one or more clinical correlates of the one or more physiosignals to output one or more predicted physiosignals.

29. The method of claim 28, wherein the one or more physiosignals received by the encoder as an input comprise a spirometric flow rate over time of an expiration, a spirometric total volume expired over time of the expiration, and a spirometric flow rate normalized to total volume expired over time of an inspiration, and wherein the one or more clinical correlates of the one or more physiosignals comprise at least one of an FEV1, an FVC, a PEF, an FEV1/FVC, or an FEF25-75%.

30. The method of claim 25, wherein the one or more latent variables output by the encoder consist of two latent variables and wherein the he one or more clinical correlates of the one or more physiosignals comprise an FEV1, an FVC, a PEF, an FEV1/FVC, and an FEF25-75%.

31. A method comprising:
obtaining a respective set of associations between each of one or more latent variables that represent variability within a set of one or more physiosignals across a population and genomic data of the population;
obtaining additional genomic data for a patient;
using the obtained sets of associations, determining a set of polygenic scores for the patient based on the additional genomic data that includes a respective polygenic score for each of the one or more latent variables;
obtaining a model that has been trained to predict a polygenic score with respect to a specified disease or condition based on a set of polygenic scores determined for the one or more latent variables;
applying the set of polygenic scores determined for the patient to the model to generate a polygenic score for the patient with respect to the specified disease or condition; and
based on the generated polygenic score for the patient with respect to the specified disease or condition, prescribing a drug to the patient, providing a treatment to the patient, or performing a diagnostic on the patient.

32.-33. (canceled)
